(19) United States
(12) Patent Application Publication
(10) Pub. No.: US 2020/0101312 A1
(43) Pub. Date: Apr. 2, 2020

(54) PHOTOTHERAPY DEVICE

(71) Applicant: GCSOL TECH CO., LTD., Taichung City (TW)

(72) Inventors: JUIFEN PAI, Taichung City (TW); CHEN PING KUO, Taichung City (TW)

(21) Appl. No.: 16/527,014

(22) Filed: Jul. 30, 2019

(30) Foreign Application Priority Data
Sep. 29, 2018 (CN) PCT/CN2018/108692

(51) Int. Cl. A61N 5/06

(52) U.S. Cl. CPC A61N 5/0616; A61N 2005/065; A61N 2005/0632; A61N 2005/0645

(57) ABSTRACT

Disclosures of the present invention describe a phototherapy device mainly comprises a phototherapeutic host and a patch module having a connection base, wherein the patch module can be assembled with the phototherapeutic host by letting the connection base be connected with the base through an interlocking mechanism, a rotary locking mechanism or a magnetic lock mechanism. In addition, the phototherapeutic host can be attached to and detached from the patch module. Briefly speaking, this phototherapy device has advantages of simple structure, small volume, being portable, being easy to be operated, and having patch units that are replaceable. In addition, the phototherapeutic host is designed to have a modular structure comprising a base, a PCB, a lighting unit, and a cover, such that the phototherapeutic host can be manufactured and/or used individually.

### BACKGROUND OF THE INVENTION

### 1. Field of the Invention

The present invention relates to the technology field of light therapy devices, and more particularly to a portable phototherapy device that has small volume and is easy to be operated. This phototherapy device can be patches on a user's skin so as to emit a light with at least one wavelength to target acupuncture point of the user, thereby achieving a light therapy in combination with a magnetic field therapy or a vibration excitation on the acupuncture point.

### 2. Description of the Prior Art

Acupuncture treatment is one kind of traditional Chinese pain relieving therapy. During a patient receiving the acupuncture treatment, a fine metal needle is adopted for an acupuncture instrument so as to be penetrated into a specific acupuncture point of a meridian system of the patient, thereby dredging vital energy (also called “qi”) and blood that flow in the meridian and collateral system. As a result, a bad circulation of vital energy and blood in the meridian system of the patient would be therefore improved, such that the meridian system is led to resume normal operations.

Because there is danger lurking in the progress of the acupuncture treatment, government does not permit everyone to be a dominator of the acupuncture treatment except for a relative medical specialist who has a professional license. On the other hand, it is known that the acupuncture treatment may cause patient suffer from certain side effect(s) or undesired accidents, like fainting during acupuncture treatment and wound infection.

More recently, light or magnetic force (field) has been adopted for replacing the fine metal needle to be an alternative acupuncture instrument for use in acupuncture treatment. Clinic experiments have found that, after operating a light therapy apparatus to emit a specific light for illuminating human skin, a series of biochemical reactions are induced in an illuminated area of the human skin, thereby activating the circulation of vital energy (also called “qi”) and blood in the meridian system. On the other hand, clinic experiments have also found that, treating human skin with a certain intensity of magnetic force (field) can also achieving the same medical effect as that resulted from the light therapy. In addition, the medical effect can be obviously enhanced when utilizing the light therapy apparatus to apply the light therapy to human skin in combination with a certain intensity of magnetic force (field).

However, owing to the fact that both the conventional light therapy apparatus and the conventional magnetic force generator have a large and complex structure, each of the two apparatuses is unportable and is not easy to be operated by any one user. For example, a user needs to enter a treatment room of a commercial van-type light therapy apparatus in order to let his whole body to be illuminated by a specific light that is provided by a light source disposed in the treatment room. The commercial van-type light therapy apparatus is commonly assembled in a specific site and cannot be moved arbitrarily due to its large apparatus volume. Although there is one kind of light therapy device having smaller volume developed for replacing the van-type light therapy apparatus, the operation ways of the small-volume light therapy device are still complicated. When operating the light therapy device, it needs to firstly set up a supporting framework on ground for supporting a light source, and then drive the light source to radiate a specific light so as to illuminate human body. Strictly speaking, the light therapy device just has a limited volume miniaturization and still has a certain thickness, causing the light therapy device cannot be portable yet.

On the other hand, a phototherapeutic device, exhibiting advantages of simple structure and can be directly patched on human skin, is disclosed in a worldwide patent application No. PCT/CN2016/085297. However, owing to the fact that a number of patch units of the phototherapeutic device only have a limited unit lifetime and are unable to be replaced, causing the phototherapeutic device also has a limited device lifetime. In despite of adding the number of the patch units is found to prolong the unit lifetime, that would simultaneously leads a whole thickness of the phototherapeutic device to be increased, thereby causing the user to feel a uncomfortable sense of wearing the phototherapeutic device. In addition, for facilitating the phototherapeutic device being patched on different parts of a user, it needs to design different appearances and/or structures for the patch units of the phototherapeutic device. However, as mentioned above, the patch units of the phototherapeutic device are non-replaceable.

Therefore, above descriptions have revealed that, how to improve or redesign the structure of the conventional phototherapeutic device has become an important issue. In view of that, inventors of the present application have made great efforts to make inventive research and eventually provided a phototherapy device that has a plurality of advantages of simple structure, small volume, being portable, being easy to be operated, and having patch units that are replaceable.

### SUMMARY OF THE INVENTION

The primary objective of the present invention is to disclose a phototherapy device that can be operated by press activation, touch control, or remote control, so as to emit a specific light to illuminate human skin. The phototherapy device mainly comprises a phototherapeutic host and a patch module, wherein the phototherapeutic host is particularly designed to have a modular structure, so as to be manufactured and/or sold individually. On the other hand, the patch module is designed to have a patch sheet (or wearing body) with a connection base, such that the phototherapeutic host can be attached to and detached from the patch sheet. By such design, it is facilitated this novel phototherapy device being patched on different parts of a user by combining the phototherapeutic host with the patch sheet (or wearing body) having correspondingly-suitable appearance or structure, thereby making the user to feel a comfortable sense of wearing the phototherapy device.

As described in detail below, the present invention discloses a phototherapy device that has replaceable patch units and an independent phototherapeutic host for long-time use. Therefore, it is able to replace the used or unhygienic patch sheet (or wearing body) by a new one, for making the user to feel a comfortable sense of wearing the phototherapy device On the other hand, this novel phototherapy device has a plurality of advantages of simple structure, small volume, being portable, being easy to be operated, and having patch units that are replaceable. The phototherapeutic host is configured to emit a light with a specific wavelength band for increasing the applications of the phototherapy device. Moreover, for the purpose of enhancing medical effect, it is able to utilize this phototherapy device to carry out a light therapy in combination with a magnetic force (field) or a vibration excitation on at least one acupuncture point of the user.

In order to achieve the primary objective of the present invention, the inventor of the present invention provides an embodiment for the phototherapy device, comprising:
a phototherapeutic host, comprising:
   a base;
   a printed circuit board (PCB), being disposed on the base, and comprising with at least one driving circuit and a plurality of electrically-conductive members for electrically connecting a power unit;
   a lighting unit, being disposed on the PCB so as to be located between the PCB and the base, and being electrically connected to the driving circuit; and
   a cover, being connected to the base for covering the power unit, the PCB, the lighting unit, and the base; and
a patch module having a connection base, wherein the patch module is assembled with the phototherapeutic host by letting the connection base be connected with the base through an interlocking mechanism, a rotary locking mechanism or a magnetic lock mechanism.

In the embodiment of the phototherapy device, wherein the base is selected from the group consisting of transparent circular-shaped base, transparent semicircular-shaped base, ring-shaped base, semiring-shaped base, ellipse-shape base, and rectangular-shaped base.

In the embodiment of the phototherapy device, wherein the driving circuit comprising a micro controller, a variable resistance and at least one electrically regulating unit, and the further comprises an auxiliary means that comprises an electric charging unit, a signal transmitting unit and a lighting mode decision unit, wherein the electric charging unit is configured for executing an electric charging process to the power unit0 by a wireless way or a wired way, and the lighting mode decision unit is adopted for editing or changing at least one lighting mode in the micro controller through the signal transmitting unit. Moreover, a controlling means is established in the micro controller by a form of firmware, function library, application (App) program, or operands, so as to control the lighting unit.

In the embodiment of the phototherapy device, wherein the patch module further has a patch sheet with a specific appearance that is particularly designed according to a variety of parts of human body, and the specific appearance is selected from the group consisting of rectangular shape, long rectangular shape, intercross shape, Y shape, butterfly wing shape, and irregular shape. In addition, the patch module can be particular designed to have a wearing body that is selected from the group consisting of vest, brassiere, wristband, and waistband.

In the embodiment of the phototherapy device, wherein the connection base is located at a position of the patch sheet, and the position is adjustable for making the connection base be located between the phototherapeutic host and the patch sheet, or for making the patch sheet be located between the phototherapeutic host and the connection base.

In the embodiment of the phototherapy device, wherein the patch sheet is made of a flexible material and provided with at least one cutting trace, and the cutting trace is propped so as to open and become an opening after the connection base is connected with the base for making the patch module be assembled with the phototherapeutic host.

For achieving the primary objective of the present invention, the inventor of the present invention provides another one embodiment for the phototherapy device, comprising:
a patch module having a connection base;
a base, being configured to be attached to or detached from the connection base;
a plate, being disposed on the patch module, and comprising a lighting unit and a coil electrically connected to the lighting unit; and
a wireless circuit board, being connected to the base, and being configured to transmit a driving power to the lighting unit through the coil.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows an exploded diagram of a first embodiment of a phototherapy device according to the present invention;

FIG. 2 shows an exploded diagram of a second embodiment of the phototherapy device according to the present invention;

FIG. 3 shows a stereo diagram for depicting a first modular structure of a phototherapeutic host of the phototherapy device;

FIG. 4 shows a stereo diagram for describing the phototherapeutic host and an auxiliary means;

FIG. 5 shows a stereo diagram for depicting a second modular structure of the phototherapeutic host;

FIG. 6 shows a stereo diagram for depicting a third modular structure of the phototherapeutic host;

FIG. 7 shows a stereo diagram for depicting a fourth modular structure of the phototherapeutic host;

FIG. 8 shows a stereo diagram for depicting a fifth modular structure of the phototherapeutic host;

FIG. 9 shows a stereo diagram for depicting a sixth modular structure of the phototherapeutic host;

FIG. 10 shows a stereo diagram for depicting a seventh modular structure of the phototherapeutic host;

FIG. 11 shows a stereo diagram for depicting an eighth modular structure of the phototherapeutic host;

FIG. 12 shows a stereo diagram for depicting a ninth modular structure of the phototherapeutic host;

FIG. 13 shows a functional block diagram of a driving circuit of the phototherapeutic host; and

FIG. 14 shows a functional block diagram of the driving circuit in combination with the auxiliary means.

### DETAILED DESCRIPTION OF THE PREFERRED EMBODIMENTS

To more clearly describe a phototherapy device disclosed by the present invention, embodiments of the present invention will be described in detail with reference to the attached drawings hereinafter.

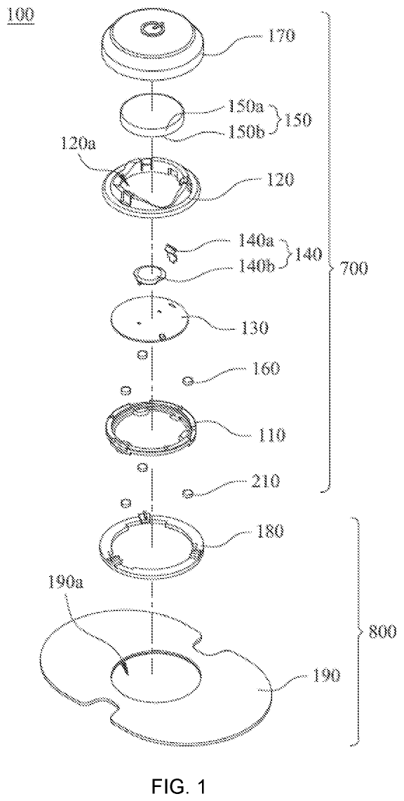

With reference to FIG. 1, there is provided an exploded diagram of a first embodiment of a phototherapy device according to the present invention. As FIG. 1 shows, the phototherapy device 100 consists of a phototherapeutic host 700 and a patch module 800, wherein the phototherapeutic host 700 can be attached to and detached from the patch module 800. In the present invention, the phototherapeutic host is particularly designed to have a modular structure comprising a base 110, a fixing means 120, a PCB 130, a plurality of electrically-conductive members 140, a lighting unit 160, a power unit 150, and a cover 170, such that the phototherapeutic host 700 can be manufactured and/or used individually. On the other hand, the patch module 800 comprising a patch sheet 190 and a connection base 180.

As described in detail below, the fixing means 120 is connected to the base 110, and both the fixing means 120 and the base 110 have a hollow structure. Moreover, the PCB 130, comprising a driving circuit the electrically-conductive members 140, is disposed in an accommodating space 120a of the fixing means 120. From FIG. 1, it is understood that the electrically-conductive members 140 comprises a positive electrically-conductive member 140a and a negative electrically-conductive member 140b.

The power unit 150 (i.e., a battery) is also disposed in an accommodating space 120a so as to electrically connect the positive electrically-conductive member 140a and the negative electrically-conductive member 140b by a positive electrode 150a and the negative electrode 150b thereof. Moreover, the lighting unit 160 is disposed on the PCB 130 so as to be located between the PCB 130 and the base 110. As FIG. 1 shows, the lighting unit 160 is electrically connected to the driving circuit, and consists of three LED components. In addition, the cover 170 is assembled with the base 110 by connecting to the fixing means 120, so as to cover the power unit 150, the PCB 130, the lighting unit 160, the fixing means 120, and the base 110.

The patch module 800 is used for patching onto a user's skin or a specific part of human body by the patch sheet 190 thereof. In the present invention, the patch module 800 is assembled with the phototherapeutic host 700 by letting the connection base 180 be connected with the base 110 through an interlocking mechanism, a rotary locking mechanism or a magnetic lock mechanism. For example, it is able to firstly dispose the connection base 180 on the patch sheet 190, and then letting the connection base 180 and the base 110 be connected to each other. Of course, by the use of the connection base 180, the patch sheet 190 of the patch module 800 can also be located between the connection base 180 and the base 110. It is worth noting that, there is some medical material coated on a surface of the patch sheet 190. When the phototherapy device 100 is used, a light therapy effect is achieved on an acupuncture point of the user, wherein the light therapy effect is made by a specific light emitted from the lighting unit 160 in combination with the medical material. Therefore, the patch sheet 190 is formed with a light passing portion 190a for the specific light. In one embodiment, the said light passing portion 190a can be made of a transparent material so as to be disposed in the patch sheet 190. Moreover, the light passing portion 190a can also be an opening directly formed on the patch sheet 190.

It is worth further explaining that, by letting the connection base 180 and/or the base 110 be made of a magnetic material, this phototherapy device 100 can be operated to achieve a light therapy on a specific part of human body in combination with a magnetic force (field).

When assembling the phototherapeutic host 700, the fixing means 120 is connected with the base 110, and the PCB 130 is subsequently disposed in the accommodating space 120a of the fixing means 120. After that, the electrically-conductive members 140 are disposed on the PCB 130. FIG. 1 depicts that the fixing means 120 is embedded into the base 110, and the power unit 150 is also disposed in the accommodating space 120a for electrically connecting the electrically-conductive members 140. Consequently, the cover 170 is connected to the base 110 for covering the power unit 150, the PCB 130, the lighting unit 160, and the base 110. Therefore, the assembly of the phototherapeutic host 700 is completed, such that the phototherapeutic host 700 has a modular structure consisting the base 110, the fixing means 120, the PCB 130, the electrically-conductive members 140, the lighting unit 160, the power unit 150, and the cover 170. Moreover, the cover 170 is also used for protecting each of the several units, and is detachable.

Beside protecting each of the several units, the cover 170 is also configured for being an activating switch of the lighting unit 160. In one embodiment, the cover 170 is made of a flexible and electrically-isolative material. Therefore, after pressing the cover 170, the power unit 150 moves to make the positive electrically-conductive member 140a and the negative electrically-conductive member 140b form a short circuit on the circuit of the PCB 130. In such case, the driving circuit is configured to drive the lighting unit 160 to emit a specific light for radiating the base 110. Of course, the driving circuit can also be used to change or modulate a light intensity and/or a wavelength band of the specific light. In one embodiment, the power unit 150 is a rechargeable battery, such as thin film battery, fuel cell or button battery. FIG. 1 shows that the negative electrically-conductive member 140b is a metal spring leaf After the pressing force applied on the cover 170 is released, the metal spring leaf would have a spring back.

The light with specific wavelength band radiated from the lighting unit 160 passes through the base 110 having a hollow structure. In a practicable embodiment, the base 110 is selected from the group consisting of transparent circular-shaped base, transparent semicircular-shaped base, ring-shaped base, semiring-shaped base, ellipse-shape base, and rectangular-shaped base. On the other hand, the patch sheet 190 has a specific appearance that is selected from the group consisting of rectangular shape, long rectangular shape, intercross shape, Y shape, butterfly wing shape, and irregular shape.

Furthermore, by letting at least one magnetic connection member 210 be provided between the base 110 and the patch sheet 190, this phototherapy device 100 can be operated to achieve a light therapy on a specific part of human body in combination with a magnetic force (field). The magnetic connection member 210 is selected from the group consisting of ball-shaped magnetic connection member, cylinder-shaped magnetic connection member and ring-shaped magnetic connection member.

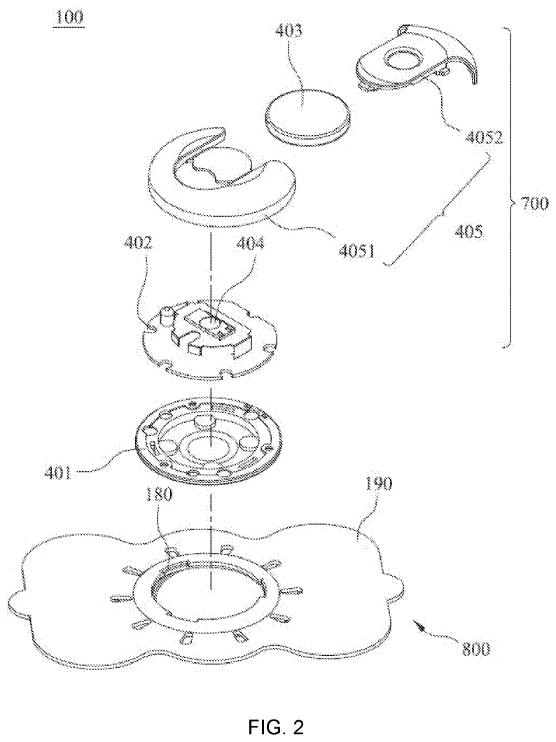

FIG. 2 shows an exploded diagram of a second embodiment of the phototherapy device according to the present invention. According to the particular design of the present invention, the phototherapeutic host 700 having modular structure can be manufactured, sold and/or used individually. In other words, each of the base 401, the PCB 402, the power unit 403, a touch switch 404, and a cover 405 is replaceable, so as to make the phototherapeutic host 700 possess different functions.

As FIG. 2 shows, the PCB 402 is connected to the base 401, and comprises driving circuit and electrically-conductive members for electrically connecting the power unit 403. On the other hand, the touch switch 401 is disposed between the PCB 402 and the base 401, and is electrically connected to the driving circuit. Moreover, the cover 405 is used for covering the PCB 402, the power unit 403, the lighting unit, and the touch switch 404. In FIG. 2, the lighting unit is not shown because it is disposed onto the bottom surface of the PCB 402. FIG. 2 also depicts that the cover 405 consists of a main body 4051 and a removable structure 4052. It is understood that, to make the cover 405 consists of the main body 4051 and the removable structure 4052 is helpful for the replacement of the power unit 403. In a nutshell, the phototherapeutic host 700 shown in FIG. 2 has a modular structure different from that of the phototherapeutic host 700 shown in FIG. 1. That means a user is able to select any one type of the phototherapeutic host 700 to be assembled with the patch module 800, so as to achieve different applications and/or functions.

Moreover, the patch module 800 can also be designed to have different structure. For example, the patch module 800 can be designed to have a patch sheet 190 or a wearing body (not shown). As FIG. 2 shows, the patch module 800 is assembled with the phototherapeutic host 700 by letting the connection base 180 be connected with the base 401. Therefore, the phototherapeutic host 700 can be attached to and detached from the patch module 800. In one embodiment, the wearing body is selected from the group consisting of vest, brassiere, wristband, and waistband.

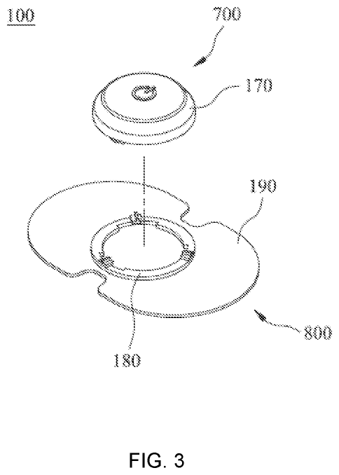

FIG. 3 shows a stereo diagram for depicting a first modular structure of the phototherapeutic host that is shown in FIG. 2. As FIG. 3 shows, the connection base 180 is disposed on the patch 190 of the patch module 800, such that the patch module 800 can be assembled with the phototherapeutic host 700 by letting the connection base 180 be connected with the base 110. As a result, the phototherapy device 100 is obtained, and can be directly patched on a user's skin for executing a light therapy and/or a magnetic therapy on at least one acupuncture point of the user. Moreover, the patch 190 of the phototherapy device 100 is replaceable.

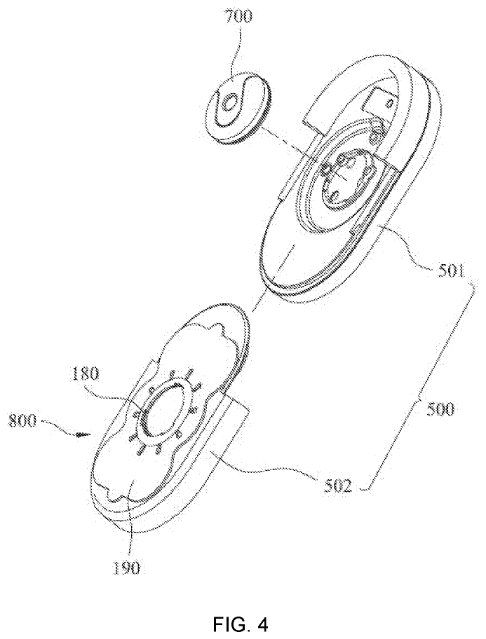

FIG. 4 provides a stereo diagram for describing the phototherapeutic host and an auxiliary means. In one embodiment, the phototherapy device 1 of the present invention further comprises an auxiliary means 500, which comprises a first functional part 501 and a second functional part 502. From FIG. 4, it is found that the phototherapeutic host 700 is disposed on the first functional part 501, and the patch module 800 is put on the second functional part 502. Therefore, by operating the auxiliary means 500, the power unit 150 of the phototherapeutic host 700 is electrically charged by the auxiliary means 500 though a wireless way or a wired way. Moreover, it is facilitated for the user to edit or change a light therapy mode of the phototherapeutic host 700 through the auxiliary means 500.

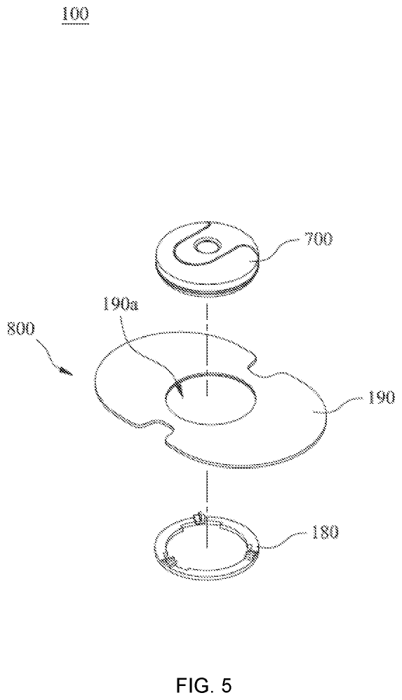

FIG. 5 shows a stereo diagram for depicting a second modular structure of the phototherapeutic host that is shown in FIG. 2. From FIG. 5, it is found that, the connection base 180 is located at a position of the patch sheet 190, and the position being adjustable for making the patch sheet 190 be located between the base 110 of the phototherapeutic host 700 and the connection base 180. In such case, there is a central opening 190a formed on the patch sheet 190, such that the connection base 180 is disposed in the central opening 190a and is connected to the base 110.

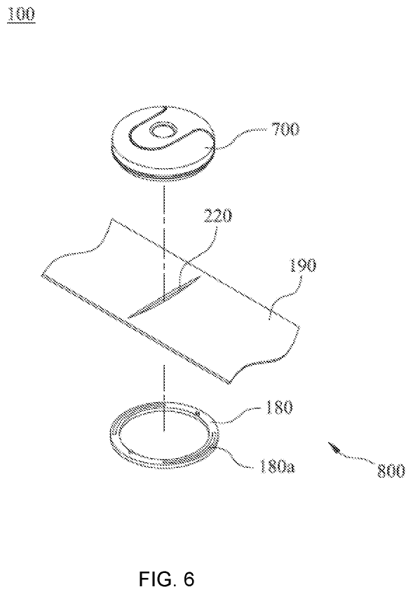

FIG. 6 illustrates a stereo diagram for depicting a third modular structure of the phototherapeutic host that is shown in FIG. 2. From FIG. 6, it is found that, the patch sheet 190 is made of a flexible material and provided with at least one cutting trace 220 thereon. It is worth noting that, after the connection base 180 is connected with the base 110 for making the patch module 800 be assembled with the phototherapeutic host 700, the cutting trace 220 is hence propped so as to open and become an opening, such that the specific light emitted from the lighting unit 160 can pass through the opening. Moreover, it can further form at least one through hole 180a on the connection base 180, so as to make two edges of the patch sheet 190 be embedded into the through hole 180a, thereby steadily fixing the patch sheet 190 on the connection base 180.

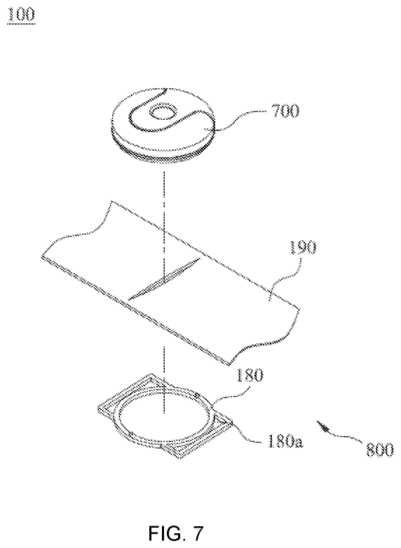

FIG. 7 provides a stereo diagram for depicting a fourth modular structure of the phototherapeutic host that is shown in FIG. 2. FIG. 7 shows that, the patch sheet 190 is made of a flexible material and provided with at least one cutting trace 220 thereon. After the connection base 180 is connected with the base 110 for making the patch module 800 be assembled with the phototherapeutic host 700, the cutting trace 220 is hence propped so as to open and become an opening, such that the specific light emitted from the lighting unit 160 can pass through the opening. Moreover, it can further form at least one through hole 180a on the connection base 180, so as to make two edges of the patch sheet 190 be embedded into the through hole 180a, thereby steadily fixing the patch sheet 190 on the connection base 180.

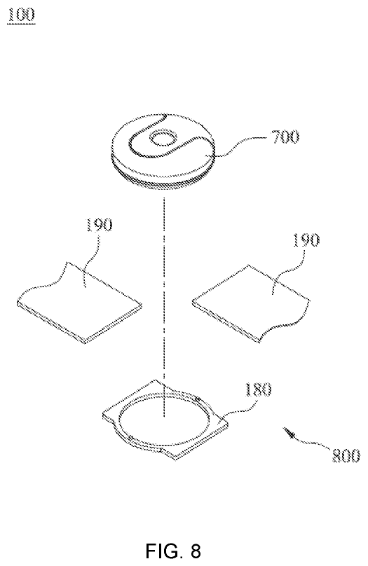

FIG. 8 illustrates a stereo diagram for depicting a fifth modular structure of the phototherapeutic host that is shown in FIG. 2. In FIG. 8, the patch module 800 has two patch sheets 190, and both the two patch sheets 190 are located between the base 110 of the phototherapeutic host 700 and the connection base 180. Moreover, each of the two patch sheets 190 has an opening that is used for allowing the specific light emitted from the lighting unit 160 to pass through.

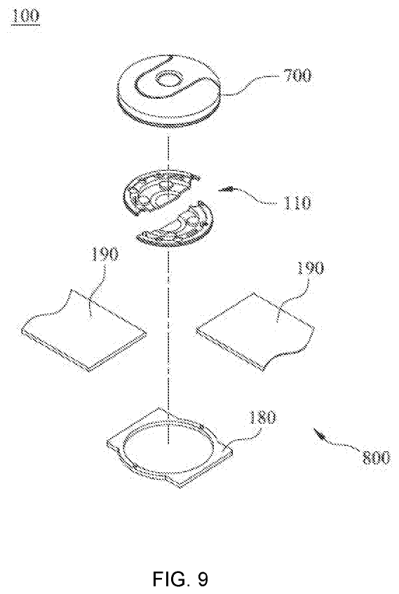

FIG. 9 shows a stereo diagram for depicting a sixth modular structure of the phototherapeutic host that is shown in FIG. 2. In FIG. 9, the patch module 800 has two patch sheets 190 and the phototherapeutic host 700 has two semicircular-shaped bases 110. The connection base 1180 with ring-shaped structure makes the two patch sheets 190 be respectively disposed between the two semicircular-shaped bases 110 of the phototherapeutic host 700 and the connection base 180. Moreover, each of the two patch sheets 190 has an opening that is used for allowing the specific light emitted from the lighting unit 160 to pass through.

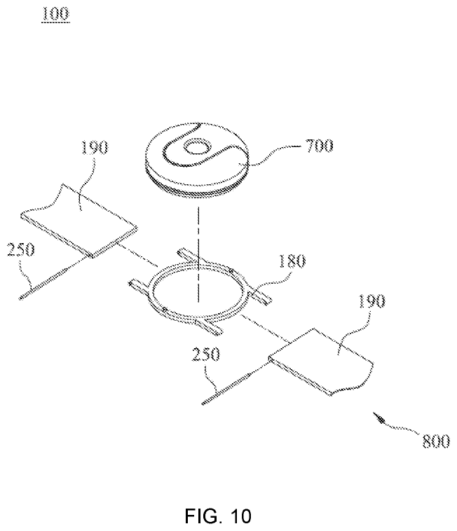

FIG. 10 illustrates a stereo diagram for depicting a seventh modular structure of the phototherapeutic host that is shown in FIG. 2. In FIG. 10, the patch module 800 has two patch sheets 190, and the patch sheets 190 are pivotally connected to two sides of the connection base 180 by two pivotal shafts 250, respectively. Therefore, the patch module 800 is assembled with the phototherapeutic host 700 by letting the connection base 180 be connected with the base 110 through an interlocking mechanism, a rotary locking mechanism or a magnetic lock mechanism.

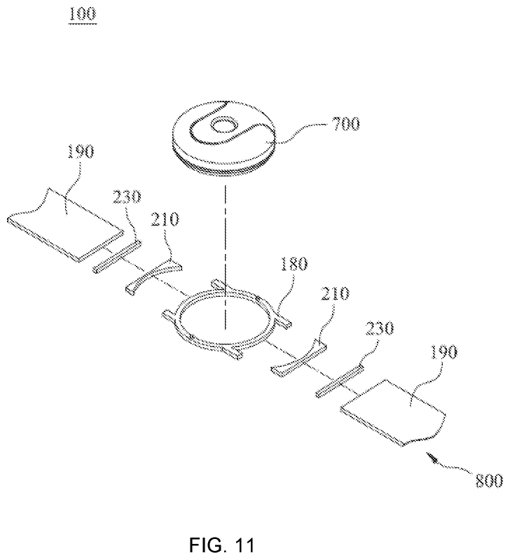

FIG. 11 shows a stereo diagram for depicting an eighth modular structure of the phototherapeutic host that is shown in FIG. 2. In FIG. 10, the patch module 800 has two patch sheets 190, and the patch sheets 190 are pivotally connected to two sides of the connection base 180 by two magnetic units, respectively. In which, each of the two magnetic units comprises a first member 210 and a second member 230. The first member 210 is made of magnetic material, and the second member 230 can be made of a magnetic material or a non-magnetic material. The magnetic material can be a ferromagnetic material comprising several elements of Fe, Co and Ni. Therefore, the patch module 800 is assembled with the phototherapeutic host 700.

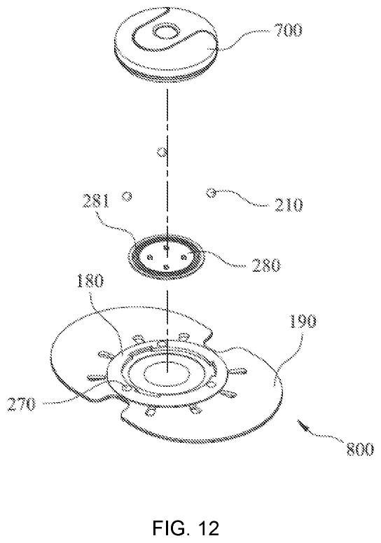

FIG. 12 provides a stereo diagram for depicting a ninth modular structure of the phototherapeutic host that is shown in FIG. 2. In FIG. 12, the PCB 130 comprises a wireless circuit thereon and a plate 208, wherein the plate 280 is connected to the patch sheet 190 and provided with several light emitting components thereon. It is worth noting that, there are three recesses 270 formed on the connection base 180, and three ball-shaped magnetic connection members (i.e., magnetic unit 210) are correspondingly disposed in the three recesses 270. It needs further explain that, the recess 270 can be further processed to have circular-shaped structure for simultaneously receiving several ball-shaped magnetic connection members. Particularly, an external electronic device like smal tphone, tablet PC and laptop PC is able to transmit a control signal to the phototherapeutic host 700 through the wireless circuit of the PCB 130, such that wireless circuit transmits a driving power to the plate 280 for driving the light emitting components to emit light. Moreover, the plate 280 is further provided with a coil 281 thereon, and the wireless circuit of the PCB 130 is configured to transmit the driving power to the light emitting components through the coil 281.

It is worth further explaining that, a number of the forgoing magnetic units 210 can be one or more, and each of the magnetic units 210 is selected from the group consisting of ball-shaped magnetic connection member, cylinder-shaped magnetic connection member and ring-shaped magnetic connection member.

Moreover, in a practicable embodiment, the patch sheet 190 can be designed to have a band structure such as a wristband or a ring-shaped structure like a bracelet, thereby facilitating a user to carrying the patch module 800.

In addition, the plate 280 is made of a flexible and waterproof material, and is disposed on the patch sheet 190. Therefore, the phototherapy device 100 does hence has properties of waterproof and flexible, such that the phototherapy device 100 is prevented from being damaged by sweat or other kinds of liquid, thereby prolonging the device lifetime of the phototherapy device 100.

Therefore, all of the embodiments of the phototherapy device 100 have clearly described. In briefly, by adjusting the disposing position of the connection base 180, it is able to make the connection base 180 of the patch module 800 be located between the phototherapeutic host 700 and the patch sheet 190. Moreover, the patch sheet 190 can also be adjusted to be located between the phototherapeutic host 700 and the connection base 180. Furthermore, the connection base 180 can also be disposed in the patch sheet 190, so as to make a connection plane of the connection base 180 and a surface plane of the patch sheet 190 have an identical horizontal level, thereby making the phototherapeutic host 700 stay at the horizontal level.

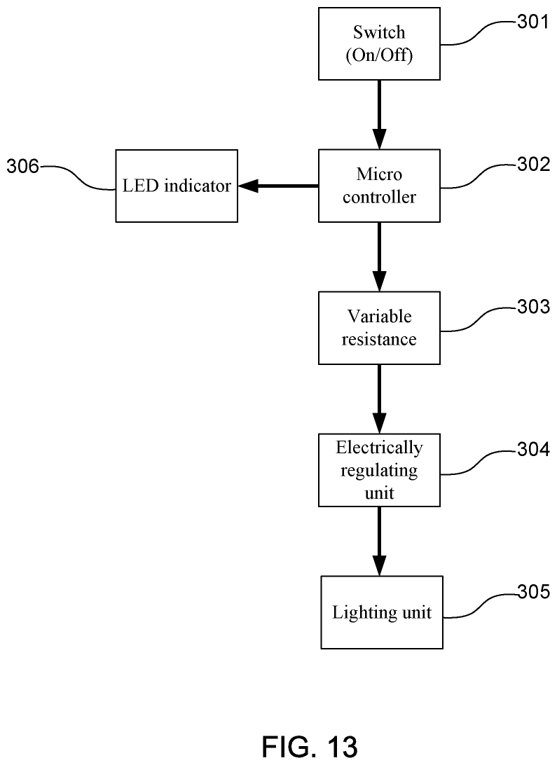

Please continuously refer to FIG. 13, which shows a functional block diagram of the driving circuit of the phototherapeutic host. In the present invention, the driving circuit mainly comprises a micro controller 302, a variable resistance 303, at least one LED indicator 30, and at least one electrically regulating unit 304 such as a current regulating unit or a voltage regulating unit. The variable resistance 303, the LED indicator 30 and the electrically regulating unit 304 are electrically connected to the micro controller 302, and the micro controller 302 is further coupled to lighting unit 305. After the switch 301 is turned ON or OFF (for example, the cover 170 is pressed or the pressing force applied on the cover is released), the micro controller 302 is activated to execute a timing control and a light control of the lighting unit 305. Moreover, the micro controller 302 is also activated to execute a vibration control in the case of the phototherapeutic host 700 further comprising a vibration module. It should know that, the variable resistance 303 and the electrically regulating unit 304 are adopted for helping the micro controller 302 to achieve of the light control of the lighting unit 305. On the other hand, the LED indicator 306 is used for showing a use state of the phototherapeutic host 700. Particularly, a controlling means is established in the micro controller 302 by a form of firmware, function library, application program (App), or operands, so as to control the lighting unit 305. Moreover, the driving circuit is also controlled by the controlling means. By such arrangement, a user is able to transmit a control signal to the wireless circuit of the PCB 130 by using an App that is installed in his smartphone, such that the wireless circuit is configured to transmit a driving signal to the lighting unit 304, there controlling the light intensity and light color of the lighting unit 304. Moreover, the user can also use the App to control a vibration intensity or a vibration frequency of the vibration module that is integrated in the phototherapeutic host 700. Briefly speaking, it is very convenient for the user to use his smartphone to control the phototherapy device 100.

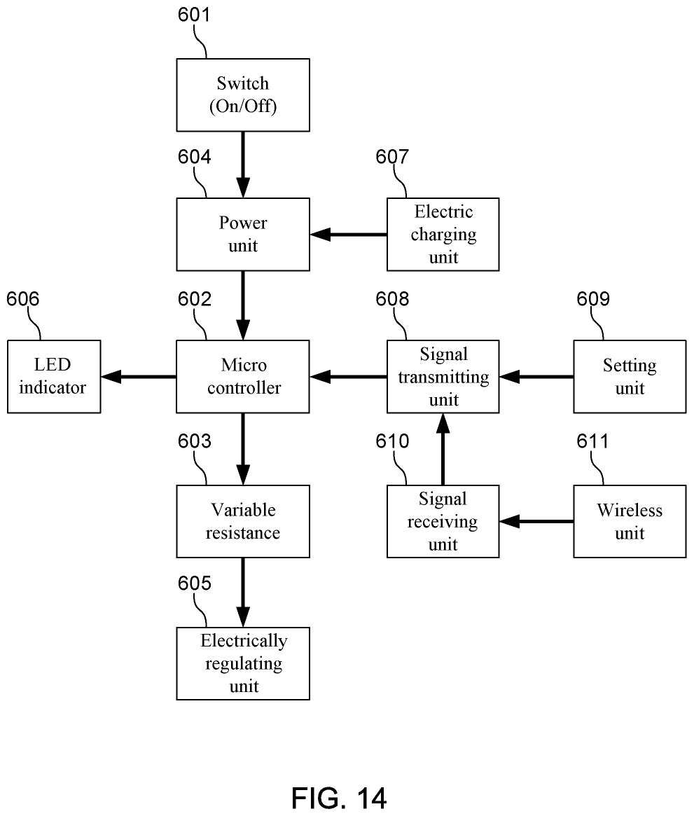

FIG. 14 shows a functional block diagram of the driving circuit in combination with the auxiliary means. As FIG. 14 shows, a system circuit constituted by the driving circuit and the auxiliary means 500 mainly comprises: switch 601, micro controller 602, variable resistance 603, power unit 604, electrically regulating unit 604 (such as a current regulating unit or a voltage regulating unit), LED indicator 606, (wireless or wired) electric charging unit 607, signal transmitting unit 608, setting unit 609, signal receiving unit 610, and a wireless unit 611. The auxiliary means 500 is configured for electrically charging the phototherapeutic host 700. Moreover, the user is able to complete a parameter setting and/or a device inspection by using the auxiliary means 500. Herein, it needs further explain that, the electric charring function, parameter setting function, signal transmitting function, and signal receiving function of the auxiliary means 500 can be respectively integrated in the electric charging unit 607, the signal transmitting unit 608, the setting unit 609, and the signal receiving unit 610. By such arrangement, a user is able to transmit a control signal to the wireless circuit of the PCB 130 by using an App that is installed in his smailphone, such that the wireless circuit is configured to transmit a driving signal to the lighting unit, there controlling the light intensity and light color of the lighting unit. Briefly speaking, it is very convenient for the user to use his smailphone to control the phototherapy device 100.

Therefore, above descriptions have completely introduced the portable phototherapy device of the present invention; in summary, the present invention includes the advantages of:

(1) It is facilitated this novel phototherapy device being patched on different parts of a user by combining the phototherapeutic host 70 with the patch sheet 190 (or wearing body) having correspondingly-suitable appearance or structure, thereby making the user to feel a comfortable sense of wearing the phototherapy device.

(2) The phototherapy device 100 of the present invention has replaceable patch unit 800 and an independent phototherapeutic host 700 for long-time use. Therefore, it is able to replace the used or unhygienic patch sheet 190 by a new one, for making the user to feel a comfortable sense of wearing the phototherapy device 100. On the other hand, this novel phototherapy device 100 has a plurality of advantages of simple structure, small volume, being portable, being easy to be operated, and having patch unit 800 that is replaceable.

The above description is made on embodiments of the present invention. However, the embodiments are not intended to limit scope of the present invention, and all equivalent implementations or alterations within the spirit of the present invention still fall within the scope of the present invention.

1. A phototherapy device, comprising:
a phototherapeutic host, comprising:
a base;
a printed circuit board (PCB), being disposed on the base, and comprising at least one driving circuit and a plurality of electrically-conductive members;
a power unit being able to be selectively and electrically connected to the plurality of electrically-conductive members;
a lighting unit, being disposed on the PCB so as to be located between the PCB and the base, and being electrically connected to the driving circuit and the power unit; and
a cover, being disposed above the power unit for covering the power unit, the plurality of electrically-conductive members, the PCB, and the lighting unit, wherein the base, the PCB, the power unit and the lighting unit are assembled together to form a modular structure; and
a patch module having a connection base, wherein the patch module is assembled with the phototherapeutic host by letting the connection base be removably connected with the base through an interlocking mechanism, a rotary locking mechanism or a magnetic locking mechanism.

2. The phototherapy device of claim 1, wherein the base is selected from the group consisting of transparent circular-shaped base, transparent semicircular-shaped base, ring-shaped base, semiring-shaped base, ellipse-shape base, and rectangular-shaped base.

3. The phototherapy device of claim 1, wherein there is at least one magnetic connection member provided on the base, and the magnetic connection member being selected from the group consisting of ball-shaped magnetic connection member, cylinder-shaped magnetic connection member and ring-shaped magnetic connection member.

4. The phototherapy device of claim 1, wherein the driving circuit comprises a micro controller, a variable resistance and at least one regulating unit electrically connected with each other, and the driving circuit is electrically connected with the lighting unit.

5. The phototherapy device of claim 4, wherein a controlling means is established in the micro controller by a form of firmware, function library, application program, or operands, so as to control the lighting unit.

6. The phototherapy device of claim 4, further comprising an auxiliary means that comprises an electric charging unit, a signal transmitting unit and a lighting mode decision unit, wherein the electric charging unit is configured for executing an electric charging process to the power unit by a wireless way or a wired way, and the lighting mode decision unit is adopted for editing at least one lighting mode in the micro controller through the signal transmitting unit.

7. The phototherapy device of claim 6, wherein the auxiliary means further comprises a signal receiving unit for receiving a control signal from an external electronic device, such that the lighting mode decision unit edits or changes the lighting mode in the micro controller according to the control signal.

8. The phototherapy device of claim 1, wherein the patch module further has a patch sheet with a specific appearance that is selected from the group consisting of rectangular shape, long rectangular shape, intercross shape, Y shape, butterfly wing shape, and irregular shape.

9. The phototherapy device of claim 1, wherein the patch module further has a wearing body that is selected from the group consisting of vest, brassiere, wristband, waistband, helmet, ear-muff, or eye cup.

10. The phototherapy device of claim 8, wherein the patch sheet is made of a flexible material and provided with at least one cutting trace thereon, and the cutting trace is propped so as to open and become an opening after the connection base is connected with the base for making the patch module be assembled with the phototherapeutic host.

11. The phototherapy device of claim 8, wherein the connection base is located at a position of the patch sheet, and the position being adjustable for making the connection base be located between the phototherapeutic host and the patch sheet, or for making the patch sheet be located between the phototherapeutic host 700 and the connection base.

12. The phototherapy device of claim 8, wherein the connection base is disposed in the patch sheet, so as to make a connection plane of the connection base and a surface plane of the patch sheet have an identical horizontal level, thereby making the phototherapeutic host stay at the horizontal level.

13. A phototherapy device, comprising:
a patch module having a connection base;
a base, being configured to be attached to or detached from the connection base;
a cover, being disposed above the base;
a plate, being disposed on the patch module, and comprising a lighting unit and a coil electrically connected to the lighting unit; and
a wireless circuit board, being connected to the base, and being configured to transmit a driving power to the coil of the lighting unit to wirelessly power and drive the lighting unit of the plate.

14. The light therapy device of claim 8, wherein the irregular shape includes mask shape matching with a face of a person, nose mask shape, eye mask shape, palm shape, foot shape.

15. The light therapy device of claim 1, wherein the cover includes a main body and a removable structure which can be removed away from the main body, resulting in an accompanying movement of the power unit.

16. The light therapy device of claim 1, wherein the phototherapeutic host includes a fixing means having an accommodating space provided therein, the fixing means is connected to the base and the PCB is disposed in the accommodating space.

17. The light therapy device of claim 1, wherein the patch module is formed into a mask shape and connected with several phototherapeutic hosts.
